(19) United States
(12) Patent Application Publication
(10) Pub. No.: US 2018/0011153 A1
(43) Pub. Date: Jan. 11, 2018

(54) MAGNETIC RESONANCE IMAGING (MRI) SYSTEM WITH ADJUSTABLE BORE ORIENTATION

(71) Applicant: Shahin Pourrahimi, Brookline, MA (US)

(72) Inventor: Shahin Pourrahimi, Brookline, MA (US)

(21) Appl. No.: 15/206,205

(22) Filed: Jul. 8, 2016

(51) Int. Cl. G01R 33/30; G01R 33/38; A61B 5/00; G01R 33/3815; A61B 5/055

(52) U.S. Cl. CPC G01R 33/307; G01R 33/3815; A61B 5/0555; A61B 5/704; G01R 33/3804

(57) ABSTRACT

A method, a system, and an article of manufacture are disclosed for obtaining imaging data from human head, jaws, sinuses, extremities and even full body, while standing, sitting or lying down. The disclosed MRI system is configured to accommodate patient shoulders in some embodiments. In various embodiments the cross section of the bore may be circular, oval, or any other appropriate and useful geometric shape. In some embodiments the body of the MRI scanner is rotatably mounted on a variable height stand to adjust for any orientation of the patient and patient's body parts.

### CROSS-REFERENCES TO RELATED APPLICATIONS

None

### TECHNICAL FIELD

This application relates generally to Magnetic Resonance Imaging (MRI). More specifically, this application relates to an adjustable bore-orientation MRI system.

### BRIEF DESCRIPTION OF THE DRAWINGS

The drawings, when considered in connection with the following description, are presented for the purpose of facilitating an understanding of the subject matter sought to be protected.

FIG. 1 shows a conventional arrangement for using a whole-body Magnetic Resonance Imaging (MRI) system for medical diagnostics;

FIG. 2 shows an example adjustable bore-orientation MRI scanner, adjusted for a patient sitting on a chair; and

FIG. 3 shows an example adjustable bore-orientation MRI scanner, adjusted for a patient leaning down.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while the following description often references cryogen-free single-bore MRI systems for imaging of human head and extremities, it will be appreciated that the disclosure may apply to other types of MRI scanners and MRI applications, such as human full body imaging, animal diagnostics and research, non-medical and/or industrial applications, and the like.

Briefly described, a method, a system, and an article of manufacture are disclosed for obtaining imaging data from human body parts in sitting, standing, bending, leaning and lying down positions. Various patient positions may bear on the physiological or physical states of his body. Thus, there may be a most suitable position of a patient for obtaining a best diagnosis. For example, scanning while standing up in a weight bearing position may reveal details and injuries and that sitting down without the force of body weight may not reveal. Similarly, with the head upright in a sitting position, the scan may reveal circulatory problems while blood is being pumped up by the heart that may not be revealed as well if the patient is lying down. Furthermore, some elderly patients, for example, may not be comfortable in certain positions.

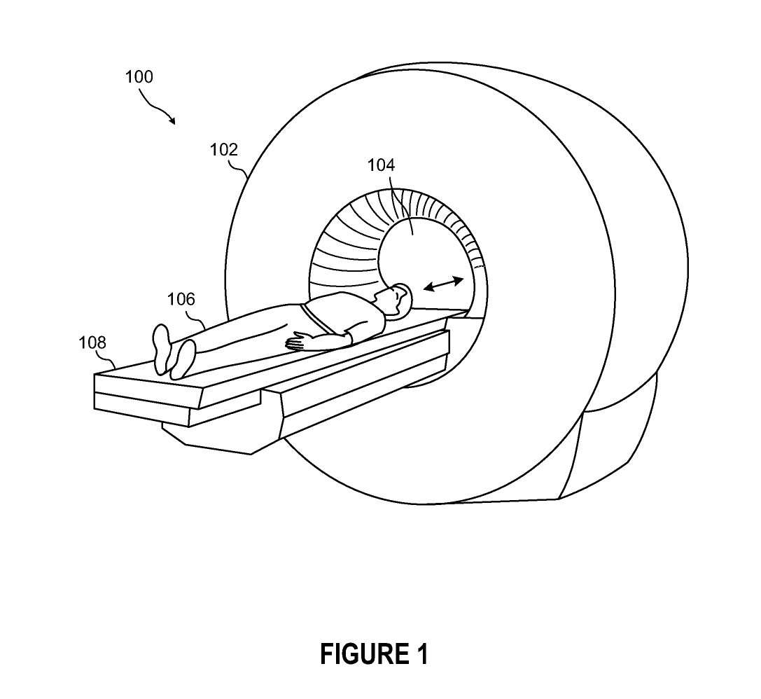

MRI is a technique for accurate and high-resolution visualization of interior of human and animal tissues. This technique is based on the nuclear magnetic resonance (NMR) property. MRI is often implemented in the form of a scanning device or scanner in which the patient lies horizontally within a scanning bore (see FIG. 1) of sufficient size to accommodate the whole body of the patient. The scanning bore is surrounded by various devices including a magnet generating a powerful static magnetic field that surrounds the patient lying within the scanning bore. The static magnetic field aligns the magnetic dipole moment of protons in atomic nuclei in the patient's tissues in the direction of the magnetic field of the magnet. Then, magnetic field gradients and Radio Frequency (RF) magnetic fields are applied to encode the protons, and generate and receive electromagnetic signals. Open MRI machines are also used for some applications in which patient is situated between two magnetic components, usually on top and bottom with open sides, instead of a cylindrical bore completely enclosing a section of the patient's body on all sides.

Common MRI scanners utilize the fact that body tissue contains a large proportion of water, and the fact that different tissue have different water contents and can be distinguished from one another. Each water molecule has two hydrogen atoms, and the nucleus of each atom has a signal spinning proton that has a positive charge. Each spinning proton has a magnetic dipole moment and is like a very small magnet that can interact with the field of other magnets. Each proton not only spins, it also precesses around its dipole directions. In ordinary condition the magnetic dipole moment direction of the protons are randomly oriented. However, when placed inside the static magnetic field of an external magnet the magnetic dipole of the protons within the body align with the magnetic field of the magnet and their precession frequency increases proportional to the external magnetic field. To get knowledge about the location of and concentration of hydrogen protons within specific tissue, a specific organ for example, the tissue/organ is placed within a highly homogeneous and uniform static field of an external magnet. In common MRI scanner the external highly homogeneous and uniform static field is between 0.2 T to 3 T. Having the proton uniformly aligned is not enough to gain knowledge about the location and concentration of the protons in specific regions of the tissue. To encode the spatial location of the protons a set of so called gradient coils are used to change the local magnetic field intensity around protons of the tissue. The set of gradient coils are charged in specific sequences and frequencies to superimpose certain linearly varying magnetic fields in X, Y, and Z direction over the static magnetic field. The gradient coils can change the field intensity and alignment of the highly homogeneous and uniform static field by, for example, 50 mT/m in the direction of the specific gradient coil being charged. So if the external highly homogeneous and uniform static field is produced over a spherical volume of 0.5 m in the diameter, then the local field, and therefore the corresponding precession frequency of the protons, at one end of the sphere is 25 mT higher than the other end, and therefore knowledge can be learned about where the protons are located because the field intensity and orientations are different at different locations. X, Y, and Z gradient coils are used to allow knowledge about proton locations three dimensionally. To produce signals from protons one or more additional coils are used to transmit and receive radio frequency electromagnetic waves pulses. The reason the additional coils pulse at radio frequency (RF) is that proton precession in external field of a fraction of a tesla to a few tesla are in RF range. When an RF coil transmits a magnetic pulse (wave) the precession of the protons are disturbed accordingly. When the transmitted pulse ends the proton dipole directions and precessions tend to return to the original orientation. The return of the dipole direction and precession of the protons produce RF signals that are received by one and the same, or different, receiving coils. The more the number of RF transmit and RF receive coils the more information about the local hydrogen protons.

The MRI image is subsequently constructed with electronic devices and computer software that process and interpret the detected RF signals.

MRI may provide better contrast between the different soft tissues of the body, such as the organs, the brain, muscles, the heart, malignant tissues, and other soft tissues compared with other imaging techniques such as Computer Tomography (CT) or X-rays. MRI is also generally safer because unlike CT scan or X-ray, no ionizing radiation is used in MRI, and thus, it is safer from a radiation standpoint. As such, MRI scanners are often used for biomedical research and diagnosis of human disease and disorder.

Imaging by an MRI scanner requires a very uniform, constant, and stable magnetic field over a specific volume. Conventionally, such a magnetic field, often referred to as a Bₒ field, is produced by a permanent or a superconducting magnet. For human applications, MRI devices that use permanent magnets typically generate a B₀ magnetic field of less than one Tesla (T). For higher resolution imaging, superconducting electromagnets producing higher magnetic fields are used. Typically, high resolution human MRI scanners use magnets that generate fields of 1 T or higher. Superconducting MRI magnets that generate a field of higher than 1 T have a cylindrical bore for equipment and patient access. Open MRI machines can also achieve 1 T, but become proportionally large, heavy, and expensive to buy, install, and operate.

If the patient bore is large enough to allow for the whole human body to fit through the scanner, it is referred to as a whole body scanner. Such scanners are large and expensive. There are certain other smaller scanners that have smaller bores, allowing the extremities, arms and legs, to fit through. These scanners, referred to as extremities scanners, are smaller and less expensive but offer acceptable scanning over arms and legs. The magnetic fields of superconducting magnets with cylindrical bores are typically generated by a number of solenoid type superconducting coils within the overall superconducting magnet.

Superconducting B₀ magnets use coils that need to be maintained at cryogenic temperatures that are lower than the critical temperature of the superconducting coils to allow superconductor mode of the coil material to appear, in which electrical resistance is zero. To achieve this, conventionally, the coils of a superconducting MRI magnet operate in a pool of liquid helium, at close to atmospheric pressure that keeps the coils at about 4.2 K.

An alternative to operating MRI superconducting coils in a pool of liquid helium is to cool the coils by a cryocooler that is physically connected to the coils by solid materials that conduct heat away from the coils. Conventionally, these types of magnets are called cryogen-free (CF) or conduction cooled magnets.

One of the customary methods of achieving a substantially constant magnetic field is to operate the superconducting magnet of an MRI system in a “Persistent Mode,” in which mode the current circulates, almost perpetually, without applying further power, through a substantially zero-resistance closed-loop set of coils. The advantage of the persistent mode is the constancy of the magnetic field, which is better than what can be achieved in a normal, driven, or non-persistent mode of operation (in which mode power is applied to maintain the current), even with the best regulated power supplies. Furthermore, in the persistent mode no additional energy is needed to power the windings and, therefore, the power supply can be turned off.

To switch the superconducting magnet from the driven mode into the persistent mode, after energizing the magnet, a “Persistent Mode Switch” may be used. For MRI magnet application a persistent mode switch is typically a non-inductive coil, or switch coil, made from special superconducting wires. When the temperature of the switch coil is below its critical superconducting temperature, the coil is in superconducting state with practically zero resistance, and when the temperature of the switch coil is above its critical temperature the switch coil is in normal (non-superconducting) state and has resistance, for example 1 to 1000 ohms. In a typical MRI superconducting magnet a suitable switch coil with proper normal state resistance is connected to the coils of the magnet such that the switch coil and the magnet coils form a closed loop.

For safety reasons, MRI scanners are used and operated within an area where the magnetic field outside of the area is less than 5 Gauss. The area inside of the 5 Gauss line is sometimes called the MRI magnet's 5-Gauss footprint. For reasons of efficiency and installation cost, superconducting magnets used in MRI applications are magnetically shielded to minimize the 5-Gauss footprint. MRI superconducting magnets may be shielded actively or passively. Actively shielded MRI superconducting magnets are often comprised of main field coils that generate the uniform static magnetic field of higher than 1 T in the area of the geometric center of the magnet systems. Another one or more shielding coils are deployed on the outside of and enclosing or surrounding the field coils to reduce the magnetic footprint of the overall magnetic system by reducing the distance from the core of the machine at which the magnetic field drops to 5 Gauss or less. The sense or direction of the electrical current in the shielding coils is opposite to the sense of the current in the field coils to induce a magnetic field that reduces or cancels the magnetic field created by the static field outside the MRI scanner. Passively shielded MRI magnets have a set of superconducting main coils and ferromagnetic materials placed strategically on the outside of the superconducting magnet to reduce external magnetic field. In various embodiments, shielding of an MRI magnet may be provided by a combination of active coils and passive ferromagnetic materials.

In an actively shield MRI superconducting electromagnet operating in persistent mode all field coils and shielding coils, as well as the persistent mode switch coil, are connected in series by superconducting electrical joints. The shield coils, however, are connected to the rest of coils such that the sense of the currents (direction of current flow) in the shielding coils are opposite those of the other coils.

FIG. 1 shows an example arrangement for using a Magnetic Resonance Imaging (MRI) system for medical diagnostics. Typically, a diagnostic arrangement 100 includes a whole body MRI scanner 102 having a scanning bore 104, which is a tunnel-like opening, to accommodate the whole body of a patient 106 lying on a patient table/bed 108. The bed 108 slides into the opening 104 to position the appropriate portion of the patient's body within the highly homogeneous area of MRI magnet system to start the scanning process.

Conventionally, all types of scans are performed with the use of whole body machines located in hospitals or outpatient clinics. Patients are required to remain motionless in a whole body machine, in recumbent position, for a considerable amount of time, even though it may be solely the head or other extremity that is being scanned. The use of whole body scanners while lying down is inevitable in most MRI applications, but in many cases a smaller adjustable bore-orientation MRI scanner can substantially benefit patients and doctors. These benefits may be more significant among elderly patients, who may be subject to considerably less discomfort, and among pediatric patients, who would benefit from lower anxiety and from the proximity of their caregivers during the procedure.

One of the advantages of the disclosed adjustable bore-orientation MRI system, as described below, is that a smaller MRI scanner may be placed in small offices without the need for a sliding patient table. Another benefit of the disclosed MRI system for patients and their doctors is that such a scanner is a point-of-care-instrument that allows for more timely diagnosis and for follow-up image evaluations during a patient's appointment at the doctor's office, rather than in a hospital setting. A cost and inconvenience associated with large full-body MRI scanners is that their size and weight precludes them from being suitable for small medical clinics, doctor's offices, and other non-hospital settings because of the special cooling, power, and housing requirements for large machines.

Additionally, the conventional full-body MRI scanner 102 that generally uses liquid helium, is large, heavy, and expensive and requires special and extensive construction and facilities including ventilation, plumbing, and safety precautions. Such large full-body MRI machines, due to their size and weight, cannot be easily moved to allow for adjustment of the bore orientation. These machines are, in most cases, fixed with respect to the floor. In some cases, large cranes are used on the outside a medical building, such as a hospital or a clinic, to lift whole body scanners and position them near the installation room. Then, sections of walls and/or windows must be temporarily removed to move the scanner inside the building and then replace the wall or window afterwards. Such moving requirements result in major expenses and inconveniences for the installation. And if the scanner needs to be moved to a different location, these laborious processes need to be repeated further adding to the overall cost of operation and ownership. Such considerations are all but absent from a smaller point-of-care machine as disclosed below. It is not required for the disclosed system to have a small MRI scanner; however, a small scanner makes the system more portable and easier to accommodate.

There are needs for MRI scanners that are: a) easier to install and operate, b) are specifically designed for a given physician's practice, c) allow for patient's comfort, and d) designed to produce the best diagnostic image based on patient's position.

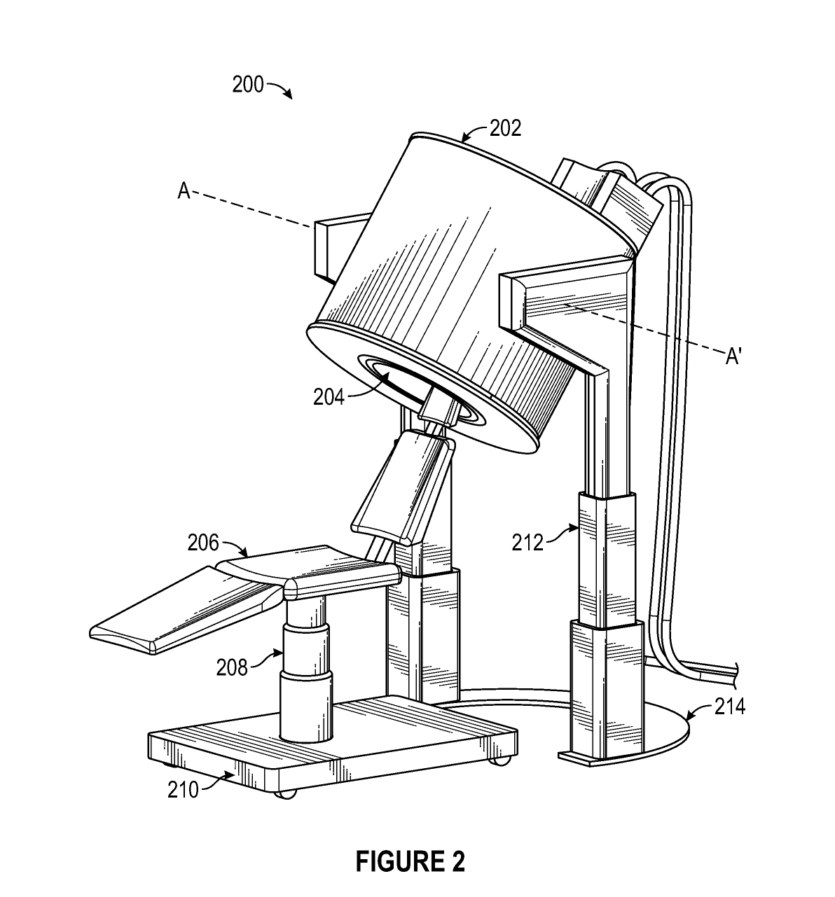

FIG. 2 shows an example adjustable bore-orientation MRI scanner, adjusted to scan the head or upper body of a patient sitting down. Typically, a diagnostic arrangement 200 includes an MRI scanner 202 having a scanning bore 204 to accommodate an extremity, such as the head, or spine of a patient sitting on chair 206. In some embodiments chair 206 may further include a height-adjustable support 208 and/or a moveable base 210. This example embodiment is configured to allow the patient's head to be in close proximity to the opening 204, shown on the front side of the scanner, to position the patient's head within the MRI magnet to start the scanning process. In the embodiment shown in FIG. 2, the back and front parts of chair 206 are adjustable and the height-adjustable support 208 is telescopic. In various embodiments, chair 206 may have fewer or more adjustable parts and the height-adjustable support 208 may use other known mechanisms to adjust the height of its seat. In some embodiments the chair may be adjusted and transformed into a narrow bed.

In any specific scanning process, a certain position of the patient may be preferable over other possible positions. For example, often it is desirable to scan patients in the positions in which they experience problems; sitting, standing, bending, leaning, as well as lying down. The disclosed system enables the patient to place himself in the position that generates the problem so that images can be acquired in that position. Correctly identifying the problem-generating pathology can markedly improve patient treatment outcomes. In addition, it enables the physician to see all the pathology he has to address

In certain situations it is preferable to scan the head and spine in lying-down position. The disclosed system can be easily and quickly adjusted for such a position as well. In yet another example the disclosed system may be adjusted for scanning a horizontally extended arm of a patient while sitting. In such a case, the height and angular orientation of the scanning bore 204 is adjusted comfortably accommodate the patient's arm while the patient is sitting on the chair 206.

FIG. 2 further illustrates a height-adjustable supporting frame 212, which holds the MRI scanner 202 that is configured to controllably and/or adjustably rotate around A-A′ axis. As shown in this example system, the height-adjustable supporting frame 212 is telescopic but in other embodiments the height-adjustable supporting frame 212 may use other known mechanisms to adjust the height of the MRI scanner 202. Shown in FIG. 2 is also an optional base 214 on which height-adjustable supporting frame 212 is attached. In some embodiments the height-adjustable supporting frame 212 may be directly attached to the floor. In yet other embodiments the base 214 may itself have wheels and be configured to be easily moved around. In such embodiments the presence of wheels also enables the supporting frame 212 to be turned around a vertical axis, if desired.

Although an adjustable chair makes it easier for the medical staff to arrange the MRI system for a scan, a mere arrangement of the height and the position of frame 212 and the angular orientation of the bore 204 can fulfill the needs of all imaging procedures.

The combination of the rotation of the MRI scanner 202 around A-A′ axis and the vertical movement of the MRI scanner 202 allows the scanning of a patient's full body or body part at any elevation while the patient is sitting, standing or lying down. The relative horizontal movements of the chair 206 and the MRI scanner 202 further add to the ease of scanning and help to alleviate any discomfort for patients. Such system allows easily positioning it in various positions and orientations as needed by the medical staff at the point and time of usage, without undue and burdensome efforts, to best serve the medical staff and the patients.

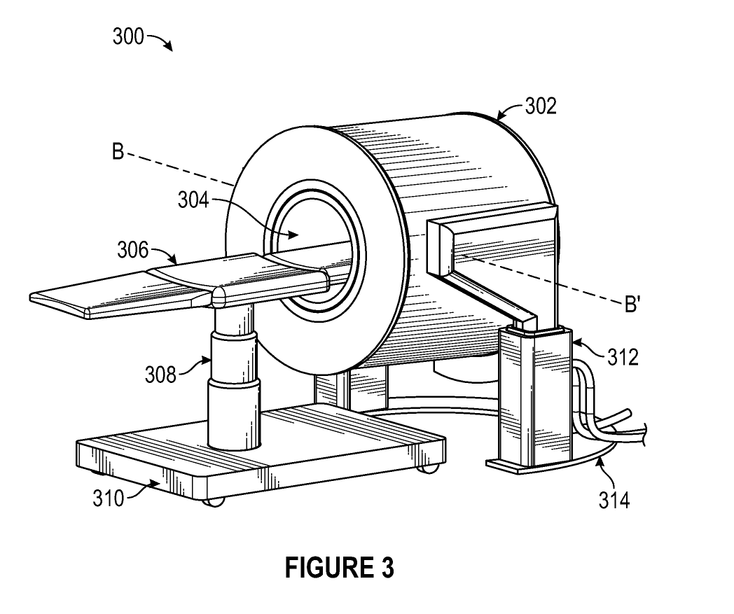

FIG. 3 shows an example adjustable bore-orientation MRI scanner adjusted for a patient lying down. The example diagnostic arrangement 300 is similar to the one illustrated in FIG. 2 and includes an MRI scanner 302 having a scanning bore 304 to accommodate an extremity, such as the head or the upper body of a patient lying down on a reclined chair 306. In some embodiments chair 306 may further include a height-adjustable support 308 and/or a moveable base 310. This example embodiment is configured to allow the patient's head to be in close proximity to the opening 304, shown on the front side of the scanner, to position the patient's head or the patient's entire upper body within the MRI magnet to start the scanning process. In the embodiment shown in FIG. 3, the back and front parts of chair 306 are adjustable and the height-adjustable support 308 is telescopic. In various embodiments, chair 306 may have fewer or more adjustable parts and the height-adjustable support 308 may use other known mechanisms to adjust the height of its seat. In general, As long as a patient's shoulder fits through the bore of the scanner most of her organs can be scanned.

FIG. 3 further illustrates a height-adjustable supporting frame 312, which holds the MRI scanner 302, which is configured to controllably and/or adjustably rotate around B-B′ axis. As shown in this example system, the height-adjustable supporting frame 312 is also telescopic but in other embodiments the height-adjustable supporting frame 312 may use other known mechanisms to adjust the height of the MRI scanner 302. Shown in FIG. 3 is also an optional base 314 on which height-adjustable supporting frame 312 is attached. In some embodiments the supporting frame 312 may be directly attached to the floor. In yet other embodiments the base 314 may itself have wheels and be configured to be easily moved, if desired. In an embodiment in which the base 314 has wheels, the supporting frame 312 may be also turned around a vertical axis if desired.

The combination of the rotation of the MRI scanner 302 around B-B′ axis and the vertical movement of the MRI scanner 302 allows the scanning of a patient's full or upper body at any elevation while the patient is sitting, standing, leaning, bending or lying down. The relative horizontal movement of the chair 306 and the MRI scanner 302 further adds to the ease of scanning and helps to alleviate any discomfort for patients.

In various embodiments, a suitable magnet for creating the static magnetic field in the MRI scanners 202 and 302 may be a Cryogen Free (CF) superconducting magnet. Notwithstanding the benefits of installation and overall economy, a CF superconducting magnet offers more conveniently the option of operating in various tilt orientations of the scanner, including the option where the scanning bore is vertical. In this case, scanning may be done on a patient in the standing position. While a CF magnet is preferred, a superconducting magnet that uses liquid helium for cooling may also be designed and manufactured to include some of the new features disclosed herein.

In various embodiments, cryocooler may be implemented using any refrigeration technique that can provide cryogenic temperatures, typically below 150 Kelvin (“K”). ThermoElectric Coolers (TEC) may be used as part of the refrigeration system. TECs, also known as Peltier coolers, are solid-state heat pumps that operate based on the Peltier effect to move heat and can create a differential temperature of up to 70° centigrade or more. The temperatures reached by a refrigeration system depend largely on material such as the refrigeration gas used, solid state junctions in TECs, and the like. Other cryogenic refrigeration systems include Gifford-Mac Mahan type systems and pulse tubes.

In various embodiments, Superconducting magnets that utilize low temperature superconductors, for example Nb—Ti and Nb₃Sn, operate at very low temperatures of 3-15 K. One method of cooling down such a superconducting magnet to these very low temperatures is by using a two-stage cryocooler (also known as a cryo-refrigerator) that makes physical contact with designated parts of the magnet system thereby extracting heat by way of conduction through the connected parts. This method of cooling is commonly referred to as being cryogen free, or conduction cooling. In these embodiments, various components of the cryostat may operate in vacuum. A conduction cooled superconducting magnet may include provisions to first cool the magnet from room temperature to its operating temperature by means other than or in addition to conduction cooling, and then maintain the temperature by conduction cooling during its normal operation. A conduction cooled superconducting magnet may include provisions to allow it to preferably deal with extra ordinary conditions like loss of electric power or emergency conditions when the magnet needs to be discharged.

Heat transfer to a superconducting magnet is by way of convection, radiation and conduction. In the case of a cryogen-free superconducting magnet, convection heat transfer is reduced by housing the superconducting magnet inside a vacuum chamber (vessel), which in this case is referred to as the cryostat. Radiation heat transfer may be reduced by housing the superconducting magnet inside a radiation shield, which in turn may be housed within the vacuum chamber. This radiation shield is cooled by the first stage of the two-stage cryocooler to a temperature of 30-60K, and is generally covered on the side facing the vacuum chamber with several layers of reflective insulation, often referred as super-insulation. Conduction heat transfer may also be reduced by proper material selection and strategic placement of such low-heat conductivity material. The radiations shield surrounds the so-called cold-mass that includes the magnet elements need to be in superconducting state, like the superconducting coils. Typically the entire cold-mass is maintained at temperatures below the critical temperature of superconducting elements. The cold-mass of cryogen-free magnet is connected to the second stage of the cryocooler to keep the cold-mass at the required low temperature.

The amount of cooling (removal of heat) that is provided by a two stage cryocooler can be a few tens of watts for the first stage achieving for example a temperature of 30-60K and a few watts for the second stage achieving 3-6K. Therefore the amount of heat transferred (also known as heat leak) to the superconducting magnet from the environment must be reduced to or be lower than the cooling capacity of the cryocooler.

Integrating a cryocooler in a conduction cooled (cryogen-free) device with the disclosed adjustable bore orientation MRI scanner, and using it instead of liquid helium, liquid nitrogen, or other cryogens to cool the coils for superconductivity, allows the size, cost, and complexity of the adjustable bore orientation MRI scanner to be reduced. Additionally, using CF magnets enables and allows various rotations and movements of the adjustable bore orientation MRI scanner.

In various embodiments, the MRI magnetic shield may be active as described above, or be passive using a natural or permanent magnet. In other embodiments, the magnetic shield may be a combination of passive and active magnets. In such configurations, the shield may be optimized to reduce the cost and size of the MRI system.

Changes can be made to the claimed invention in light of the above Detailed Description. While the above description details certain embodiments of the invention and describes the best mode contemplated, no matter how detailed the above appears in text, the claimed invention can be practiced in many ways. Details of the system may vary considerably in its implementation details, while still being encompassed by the claimed invention disclosed herein.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the claimed invention to the specific embodiments disclosed in the specification, unless the above Detailed Description section explicitly defines such terms. Accordingly, the actual scope of the claimed invention encompasses not only the disclosed embodiments, but also all equivalent ways of practicing or implementing the claimed invention.

The above specification, examples, and data provide a complete description of the manufacture and use of the composition of the invention. Since many embodiments of the invention can be made without departing from the spirit and scope of the invention, the invention resides in the claims hereinafter appended. It is further understood that this disclosure is not limited to the disclosed embodiments, but is intended to cover various arrangements included within the spirit and scope of the broadest interpretation so as to encompass all such modifications and equivalent arrangements.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. An adjustable bore-orientation Magnetic Resonance Imaging (MRI) scanner system comprising:
an MRI scanner body having at least one bore deployed therein;
a height-adjustable stand, a first end of which is placed on a floor and to a second end of which the MRI scanner body is rotatably mounted, wherein an axis of rotation of the MRI scanner body is substantially perpendicular to a longitudinal centerline of the bore; and
wherein the MRI scanner body travels upward and downward by adjusting a height of the height-adjustable stand.

2. The system of claim 1, wherein a field magnet of the system is Cryogen Free (CF).

3. The system of claim 2, further comprising a cryocooler device configured to cool the field magnet.

4. The system of claim 1, further comprising a moveable height and position adjustable chair that is configured to be optionally transformed into a narrow bed.

5. The system of claim 1, wherein the stand is configured to be manually moved on the floor.

6. The system of claim 1, wherein a field magnet of the system is surrounded by a shield magnet and the field magnet and the shield magnet are superconducting magnets configured to produce a stable and constant magnetic field or wherein the shield magnet is at least partially passive.

7. The system of claim 1, wherein coils of a field magnet of the system and a shield magnet of the system are connected in series and operate in persistent mode.

8. The system of claim 4, wherein the stand and/or the chair move upward and downward by telescopic mechanisms.

9. The system of claim 1, wherein a combination of height and angular arrangement of the scanner body accommodates any body part of a patient while sitting, standing or lying down.

10. The system of claim 1, wherein the height and angular position of the scanner body is adjusted automatically, manually, or remotely using pneumatic, hydraulic, magnetic, mechanical or electrical actuators.

11. A method of scanning human and animal body parts and organs, the method comprising:
using an adjustable bore-orientation Magnetic Resonance Imaging (MRI) scanner system having an MRI scanner body with at least one bore deployed therein, wherein the MRI scanner body is rotatably mounted on a height-adjustable stand and wherein an axis of rotation of the MRI scanner body is substantially perpendicular to a longitudinal centerline of the bore;
adjusting height and angular position of the scanner body to accommodate a body part of a patient while the patient is sitting, standing or lying down;
placing the body part to be scanned in the bore; and
scanning the body part to be scanned.

12. The method of claim 11, wherein a field magnet of the scanner system is Cryogen Free (CF).

13. The method of claim 12, further comprising a cryocooler device configured to cool the field magnet.

14. The method of claim 11, further comprising a moveable height and position adjustable chair.

15. The method of claim 11, wherein the stand is configured to be manually moved on the floor.

16. The method of claim 11, wherein a field magnet of the system is surrounded by a shield magnet and the field magnet and the shield magnet are superconducting magnets configured to produce a stable and constant magnetic field or wherein the shield magnet is at least partially passive.

17. The method of claim 11, wherein coils of a field magnet of the system and a shield magnet of the system are connected in series and operate in persistent mode.

18. The method of claim 11, wherein the height and angular position of the scanner body is adjusted automatically, manually, or remotely using pneumatic, hydraulic, magnetic, mechanical or electrical actuators.

19. An adjustable Magnetic Resonance Imaging (MRI) scanner system comprising:
an MRI scanner body having at least one bore deployed therein;
a height-adjustable stand over which the MRI scanner body is rotatably mounted;
a height and position adjustable chair on which patients sit or lie to be scanned; and
wherein a distance between the chair and the stand is adjustable.

20. The system of claim 19, wherein a back rest of the chair is adjustable, a foot rest of the chair is adjustable, the chair is configured to be moves around on wheels or rails, the chair is configured to rotate around a vertical axis, the stand is configured to be moves around on wheels or rails, and/or the stand is configured to rotate around a vertical axis, or any combination thereof.
